(19) United States
(12) Patent Application Publication
(10) Pub. No.: US 2006/0140908 A1
(43) Pub. Date: Jun. 29, 2006

(54) Methods for inducing an immune response via oral administration of an adenovirus

(76) Inventor: Hildegund C. J. Ertl, Philadelphia, PA (US)

Correspondence Address: LICATLA & TYRRELL P.C., 66 E. MAIN STREET, MARLTON, NJ, 08053, US

(21) Appl. No.: 10/559,603

(22) PCT Filed: Jun. 18, 2004
(86) PCT No.: PCT/US04/19603

§ 371 Date: Feb. 13, 2006

Related U.S. Application Data
(60) Provisional application No. 60/479,425, filed on Jun. 18, 2003.

(51) Int. Cl. A61K 48/00; C12N 15/861

(52) U.S. Cl. 424/93.2; 435/456

(57) ABSTRACT

The present invention provides a method for inducing an immune response to an adenovirus in a subject which has been pre-exposed to an adenovirus or adenoviral vector. The invention further provides a method for inducing a mucosal immune response to an antigen. The methods of the invention are carried out by oral administration of adenoviral vectors.

This application claims the benefit of priority from U.S. Provisional Application Ser. No. 60/479,425, filed on Jun. 18, 2003 whose contents is incorporated herein by reference in its entirety.

### INTRODUCTION

This invention was made in the course of research sponsored by the National Institutes of Health (NIAID Grant No. P01A1052271). The U.S. government may have certain rights in this invention.

### BACKGROUND OF THE INVENTION

Vaccines remain an efficacious medical intervention to reduce mortality and morbidity due to pathogens. While more than 400 distinct viruses can cause symptomatic infections in humans, prophylactic vaccines are available for only a fraction of these pathogens.

Traditionally, vaccines have been developed by inactivation or attenuation of pathogens. Advances in molecular biology now allow for the generation of recombinant subunit vaccines based on different carriers, which impact the magnitude and the type of the immune response to the vaccine antigen. The type of the vaccine vehicle also imposes constraints on the potential routes of vaccine delivery.

Adenoviral (Ad) recombinants of the human serotype 5 (Hu5) are efficacious as vaccine carriers in experimental animals (He, et al. (2000) Virology 270:146-161; Moraes, et al. (2002) Vaccine 20:1631-1639; Shiver, et al. (2002) Nature 415:331-335; Sullivan, et al. (2000) Nature 408:605-609; Tims, et al. (2001) Vaccine 18:2804-2807; Xiang, et al. (1996) Virology 219:220-227) and are now in clinical trials (Mincheff, et al. (2001) Crit. Rev. Oncol. Hematol. 39:125-132) and have been administered by various routes (Vos, et al. (2001) J. Gen. Virol. 82:2191-2197). Intranasal application of such vaccines has been tested (Gogev, et al. (2002) Vaccine 20:1451-1465; Xiang and Ertl (1999) Vaccine 17: 2003-2008) and shown to induce antibody responses at mucosal surfaces, the most common port of entry for most viral pathogens. Replication-defective or replication-competent Ad recombinants of human, or porcine serotypes have been demonstrated to induce cellular and humoral immunity to the target antigen upon oral or enteric administration (Hammond, et al. (2001) Arch. Virol. 146:1787-1793; Mutwiri, et al. (2000) Vaccine 19:1284-1293; Sharpe, et al. (2002) Virology 293:210-216; Vos et al. (2001) J. Gen. Virol. 82:2191-7). Further, oral administration of an AdHu5 vaccine expressing rabies glycoprotein G overcomes immunity against canine adenovirus in fox (Vos, et al. (2002) supra). Epicutenous application through dermal patches have been used, however with limited success (Lees, et al. (2002) Vet. Microbiol. 85:295-303; Shi, et al. (2001) J. Virol. 75:11474-11482).

Vaccine carriers that achieve protective immune responses upon oral immunization are needed for several reasons. Vaccines that can be given through the oral route are highly desirable for developing countries where a lack of skilled medical personnel and insufficient resources causes logistic problems for mass vaccinations given by injection. Repeated use of unsterile needles can lead to inadvertent spread of other human pathogens such as HIV-1, thus negating the benefit of vaccination (Jodar, et al. (2001) Vaccine 19:1594-1605). In developed countries facing an increased risk of intentional release of pathogens (Gostin, et al. (2002) J. Am. Med. Assoc. 288:622-628), oral vaccines would allow for a more rapid mass vaccination than could be achieved by vaccines applied by injection or by propulsion devices. In addition, mucosal vaccination such as intranasal or oral vaccination favors the induction of antibodies secreted at mucosal surfaces (Xiang and Ertl (1999) supra), which are common ports of entry for the invasion of many pathogens including those that spread through aerosoles or by sexual contact. While intranasal vaccination is cumbersome and difficult to dose, oral vaccination has proven highly successful in the poliovirus eradication campaign (Sabin (1965) J. Am. Med. Assoc. 194:872-876). Further, U.S. Pat. No. 6,348,450 discloses inducing an immune response to an adenovirus vector-encoded antigen by topically administrating an adenoviral vector. While this patent indicates that an adenovirus may be administered orally, the teachings primarily focus on administration of an adenovirus to external skin surfaces and the oral and nasal cavities.

Vaccines to viral pathogens, which can be distributed rapidly to large segments of a susceptible population, are needed. The present invention meets this long-felt need.

### SUMMARY OF THE INVENTION

The present invention relates to a method for inducing an immune response in a subject pre-exposed to an adenovirus. The method involves orally administering to a subject, that has been exposed to a first adenovirus, either by natural infection or upon administration of a vaccine such as a vaccine to adenovirus or a vaccine to another pathogenic entity based on an adenoviral vaccine carrier, an effective amount of a second adenovirus so that an immune response to a transgene product encoded for by the second adenovirus is induced. In one embodiment, the first adenovirus encodes the same transgene product as the second adenovirus. In another embodiment, the first adenovirus and second adenovirus encode different transgene products. In yet another embodiment, the transgene product is an antigenic epitope or protein from a cancer cell, virus, fungus, bacterium, protozoa, mycoplasma, or is an aberrant protein. In a further embodiment, the first adenovirus is a wild-type virus and the second adenovirus is a part of a vaccine. In an alternative embodiment, the first adenovirus and the second adenovirus are both part of a vaccine. In further embodiments, the second adenovirus, or the first adenovirus and/or second adenovirus further encode an adjuvant.

The present invention also relates to a method for inducing an immune response to a transgene product by oral priming with an effective amount of a first adenoviral vector encoding for a transgene product and subsequently systemically boosting with an effective amount of a second adenoviral vector encoding for said transgene product. In one embodiment, the transgene product is an antigenic epitope or protein from a cancer cell, virus, fungus, bacterium, protozoa, mycoplasma or is an aberrant protein. In another embodiment, the first adenoviral vector and second adenoviral vector are part of a vaccine. In a further embodiment, the first adenoviral vector and/or second adenoviral vector further encode an adjuvant. The present invention further relates to a method for inducing an immune response in an infant by orally administering to an infant an effective amount of an adenoviral vector encoding a transgene product so that an immune response to the transgene product is induced. In one embodiment, the transgene product is an antigenic epitope or protein from a virus, fungus, bacterium, protozoa, mycoplasma or is an aberrant protein. In another embodiment, the adenoviral vector is part of a vaccine. In a further embodiment, the adenoviral vector further encodes an adjuvant. The present invention also relates to a method for inducing a mucosal immune response to an antigen. The method involves orally administering an effective amount of a first adenoviral vector containing nucleic acid sequences encoding an antigen, and orally administering an effective amount of a second adenoviral vector containing said nucleic acid sequences encoding said antigen, so that a mucosal immune response to said antigen is induced. In one embodiment, the transgene product is an antigenic epitope or protein from a virus, fungus, bacterium, protozoa, mycoplasma or is an aberrant protein. In another embodiment, the first adenoviral vector encodes the same transgene product as the second adenoviral vector. In an alternative embodiment, the first adenoviral vector and second adenoviral vector encode different transgene products. In another embodiment of the present invention, the first adenoviral vector and the second adenoviral vector are homologous. In an alternative embodiment, the first adenoviral vector and the second adenoviral vector are heterologous. In a further embodiment, the first adenoviral vector and second adenoviral vector are part of a vaccine. In a further embodiment, the first adenoviral vector and/or second adenoviral vector further encode an adjuvant.

### DETAILED DESCRIPTION OF THE INVENTION

Rabies virus, a simple RNA virus, is well-defined and neutralizing antibodies against the viral glycoprotein, are known (Sullivan, et al. (2000) supra). Animal models, including those based on rodents, are considered valid for pre-clinical vaccine testing and have been used to evaluate novel vaccine carriers or adjuvants that aim to induce neutralizing antibody responses to the vaccine antigen (Xiang and Ertl (1999) supra; Xiang, et al. (2002) J. Virol. 76:2667-2675; Xiang, et al. (1994) Virology 199:132-140; Xiang, et al. (1996) supra). To reflect the genetic diversity of the human population, the studies provided herein used outbred ICR mice in addition to better-characterized inbred strains of mice.

AdHu5 virus, the most commonly used vector for pre-clinical vaccine studies is a ubiquitous pathogen, and circulating serotype-specific neutralizing antibodies found in up to 45% of the adult United States population interfere with the efficacy of systemically delivered Ad vaccines based on the homologous serotype (Farina, et al. (2001) J. Virol. 75:11603-11613; Moffatt, et al. (2000) Virology 272:159-167; Papp, et al. (1999) Vaccine 17:933-943; Xiang, et al. (2002) supra). An alternative vector system based on an Ad virus originating from the lymph nodes of a chimpanzee was developed. E1-deleted recombinants derived from this virus, designated chimpanzee serotype 68 (AdC68), induce in rodents upon systemic or intranasal application a transgene product-specific antibody response, which is not impacted by pre-existing immunity to common human serotypes of Ad virus (Xiang, et al. (2002) supra). These studies were conducted in a mouse rabies virus model considered an appropriate model for human rabies vaccines. Current vaccine lots are analyzed by a potency test in rodents (Fitzgerald, et al. (1978) Dev. Biol. Stand. 40:183-186), before release for use in humans. Rabies virus is a simple RNA virus encoding five antigens. Of those, the glycoprotein is the sole target of virus neutralizing antibodies (VNAs), which provide protection to viral challenge (Xiang, et al. (1995) Virology 214:398-404).

It has now been found that oral delivery (os) of E1-deleted Ad vectors of the serotypes Hu5 and C68 expressing the glycoprotein of the fixed Evelyn Rokitniki Abelseth (ERA) strain of rabies virus stimulate a systemic and mucosal antibody response and protection to a severe rabies virus challenge. In addition, it was found that transgene product-specific humoral immune response to oral Ad vaccination was not strongly impaired by pre-existing antibodies to the vaccine carrier and this response could be boosted by a second dose of the homologous vaccine carrier again given per os.

E1-deleted Ad vectors of human and simian serotypes induce transgene product-specific serum antibodies upon oral application. The rabies virus glycoprotein (rab.gp) was used to test for the induction of antibodies by Ad recombinants based on the human serotype 5 (AdHu5rab.gp) or the chimpanzee serotype 68 (AdC68rab.gp). It is known that upon subcutaneous (s.c.) or intramuscular (i.m.) immunization both vaccines stimulate antibodies to rabies virus, although serum titers are markedly higher upon vaccination with the AdHu5rab.gp vector. Upon intranasal (i.n) immunization, both vaccines induce more comparable titers of rabies virus-specific antibodies in sera (Xiang and Ertl (1999) supra). Thus, oral immunization of outbred ICR and inbred C57Bl/6 mice with escalating doses of the AdHu5rab.gp and the AdC68rab.gp vectors was evaluated. In general, the latter strain of mice mounts a less vigorous B-cell response to the rabies virus glycoprotein compared to other mouse strains such as ICR or C3H/He mice. The results of these studied showed mice of either strain developed antibody titers to rabies virus at doses of or above 2×10⁶ pfu. Oral immunization was not as effective as i.m. vaccination for the AdHu5rab.gp vector applied at high (10⁷ pfu) or low (10⁵ pfu) doses to groups of ICR mice.

Sera from ICR mice orally vaccinated with AdHu5rab.gp or AdC68rab.gp were tested for rabies virus-specific VNAs which are important for protection against virus infection (Xiang, et al. (1995) supra). Both vaccines induced serum VNA responses to rabies virus upon oral application and correspondingly protective immunity to rabies virus challenge given directly into the central nervous system. VNA titers and protective immunity, unlike titers tested by ELISA, showed a dose response curve for both vaccines. Upon systemic immunization with recombinant vaccines, titers detected by ELISA correlate with those determined by neutralization assays (Xiang, et al. (2002) supra; Xiang, et al. (1996) supra; Xiang, et al. (1994) Virology 199:132-140). These results indicate that antibodies elicited against the vector-encoded viral protein were directed against epitopes expressed on correctly folded protein and that these antibodies possess neutralizing activity. Upon oral immunization, this correlation was less rigorous indicating that the B-cell response targeted, in part, unfolded or partially degraded rabies virus glycoprotein, resulting in a high fraction of non-neutralizing antibodies that were detected by the ELISA. While both vaccines had reduced efficacy via oral immunization compared to systemic routes of immunization, complete protection could be achieved with either vaccine upon oral application of 2×10⁷ pfu of the vectors.

It has been shown that, upon s.c. immunization, the Adhu5rab.gp virus induced a mixed Th1/Th2 response with a ratio of IgG2a/IgG1 of approximately two, while the AdC68rab.gp virus favored stimulation of a Th1 response providing a ratio of IgG2a/IgG1 of approximately 10 (Xiang, et al. (2002) supra). This difference in isotype distribution of the transgene product-specific antibodies is not observed upon intranasal immunization (Sharpe, et al. (2002) Virology 293:210-216) or upon oral vaccine application, as the results herein indicate.

Upon intramuscular injection of the Ad recombinants, lymph nodes draining the injection sites rapidly, within less than 24 hours, acquire transgene product-expressing cells with morphological and phenotypic characteristics of mature dendritic cells. These cells may become infected at an immature stage at the site of inoculation and then upon maturation migrate to lymphatic tissues where they present the antigen to naïve T-cells. To determine which lymphatic tissues became infiltrated by recombinant Ad virus-infected migratory cells and thus were likely to participate in induction of an immune response upon oral administration of the Ad vectors, mice were fed 1×10⁸ pfu of AdHu5rab.gp or AdC68rab.gp virus. Lymph nodes (cervical and mesenteric) and Peyers' Patches were harvested at 18, 48 or 72 hours after administration, RNA was isolated and subsequently reverse transcribed and PCR-amplified for rabies virus glycoprotein- and GAPDH-specific cDNA. Rabies virus-specific amplicons were detected in all of the lymph nodes at least one of the time points, indicating that the vaccines had been taken up within the oral cavity as well as within the intestine.

It was found that E1-deleted adenoviral vaccines induce mucosal antibody responses upon oral application. Mucosal immunization such as through the oral or respiratory routes favors induction of antibodies secreted at mucosal surfaces. This was further analyzed by feeding ICR mice either the AdHu5rab.gp or the AdC68rab.gp vaccine. With oral vaccination of either vaccine, antibodies titers to rabies at vaginal mucosa and in fecal suspensions were comparable in outbred ICR mice. C57BL/6 mice generated antibodies in vaginal secretions upon oral vaccination with the AdHu5rab.gp vaccine. In contrast, oral application of the AdC68rab.gp vaccine at all doses teasted induced only low levels of mucosal antibodies in C57BL/6 mice, although these mice developed substantial serum antibody titers with the AdC68rab.gp vaccine.

Oral vaccination overcame interference by pre-existing neutralizing antibodies to the Ad vaccine carrier. Serum antibody response to the rabies virus glycoprotein when presented by a systemic AdHu5rab.gp vaccine is strongly reduced while the antibody response to a systemic AdC68rab.gp vaccine is not impaired in mice pre-exposed to AdHuS virus (Sharpe, et al. (2002) supra). Thus, it was determined whether the humoral response elicited by oral vaccination was able to overcome pre-existing immunity to the vaccine carrier. Mice were immunized with replication competent (in their natural host) AdHu5 virus given at 5×10¹¹ virus particles i.m. or at a lower dose of 5×10¹⁰ virus particles intranasally, the natural route of infection of humans by this virus. Serum antibody titers to the AdHu5 vector, tested four weeks later by a neutralization assay were ˜1:160 in the i.m. vaccinated group, which is comparable to titers commonly found in human adults. Intranasally vaccinated mice had neutralizing antibody titers below 1:20 although antibodies to Ad virus could readily be detected by ELISA. AdHu5-immune, as well as naïve mice, were subsequently vaccinated with the AdHu5rab.gp vector given either per os or i.m. Mice were bled two weeks later and serum antibody titers to rabies virus were determined by a neutralization assay. The antibody response to i.m. vaccination with the AdHu5rab.gp vector, which induced a potent response in naïve ICR mice, was completely abrogated in mice pre-exposed by i.m. inoculation with AdHu5 virus and moderately decreased from 490 to 160 IU upon intranasal pre-exposure. It has been demonstrated that even very low levels of neutralizing antibodies strongly inhibit gene transfer of E1 deletion AdHu5 vectors (Kurlyama, et al. (1998) Anticancer Res. 18:2345-2352), which is similar to the findings disclosed herein showing that mice with titers of less than 1:20 still showed a reduction in the transgene product-specific antibody response upon i.m. application of the AdHu5rab.gp vector. Antibody titers to rabies virus generated upon oral immunization were overall lower in naïve mice compared to those achieved by i.m. immunization. In mice pre-immunized systemically with AdHu5 virus prior to per os vaccination with the Adhu5rab.gp vector, VNA titers to rabies virus were identical to those elicited in mice that had not been pre-exposed to the vaccine. Pre-exposure through the airways caused a slight increase in VNA titers elicited by oral vaccination. Overall these data indicate that the efficacy of oral immunization is relatively unaffected by pre-existing neutralizing antibodies to the vaccine carrier. Part of the experiment was repeated using different doses of the AdHu5rab.gp vector to determine whether low vaccine doses could be inhibited by pre-existing antibodies to the vaccine carrier. Intranasal pre-exposure was chosen for these experiments as this regimen is better suited to induce mucosal antibodies of the IgA isotype (Xiang, et al. (1999) supra). Intranasally, pre-exposed and naïve mice were vaccinated orally with the AdHu5rab.gp vector. Two groups of mice were vaccinated with an intermediate dose of the AdC68rab.gp vector. Pre-exposure to AdHu5 virus had no effect on the transgene product-specific antibody titers in sera or at vaginal surfaces induced by the two higher doses (2×10⁶ and 2×10⁷ pfu per mouse) of the AdHu5rab.gp vector or the intermediate dose (2×10⁶ pfu) of the AdC68rab.gp vaccine. The serum and vaginal antibody response to the lowest dose (2×10⁵ pfu per mouse) of AdHu5rab.gp vaccine was marginally reduced in AdHu5 pre-exposed mice, indicating that low-dose oral immunization may be affected by mucosal pre-exposure to AdHu5 virus. The vaginal response to the highest dose of the AdHu5rab.gp vector, as well as to the intermediate dose of the AdC68rab.gp vector, was slightly increased in pre-immune mice indicating a potential benefit from the AdHu5 pre-exposure on the transgene product-specific mucosal B-cell response to the vaccine antigen. The VNA response in these studies, similar to the dose titration experiments, showed no strict correlation with the serum antibody titers determined by ELISA. Again, VNA titers were not strongly reduced in AdHu5 pre-exposed mice vaccinated with the high or intermediate doses of AdHu5rab.gp or AdC68rab.gp virus; the response to the intermediate dose of AdHu5rab.gp virus was slightly increased by approximately 2-fold which was within the range of assay variability. The isotype profiles of serum and vaginal antibodies to rabies virus were not affected in AdHu5rab.gp vector-fed mice pre-exposed to airway-administered AdHu5 virus. The isotype profile of vaginal antibodies to rabies virus elicited by oral administration of the AdC68rab.gp vaccine was shifted towards IgA in AdHu5 pre-immune mice, indicative of an AdHu5-specific T-helper cell effect on the vaccine-induced B-cell response.

Oral booster immunization enhances the antibody response to the transgene product of Ad vectors. A second oral and intermediate dose (2×10⁶ pfu) of either AdC68rab.gp or AdHu5rab.gp virus was administered to groups of ICR mice. Mice were boosted 4 weeks later orally with the same dose of homologous or heterologous carrier used for priming. Serum antibody responses to rabies virus glycoprotein were analyzed 2 and 8 weeks later. The oral booster immunization enhanced serum antibody responses at both time points. The AdHu5rab.gp-primed group responded with a similar increase in rabies virus-specific antibody titers to booster immunization with homologous or heterologous vaccine carrier as shown by ELISA. VNA titers indicated an advantage for the homologous booster immunization. In AdC68rab.gp-primed mice, booster immunization with the heterologous AdHu5rab.gp vector resulted in slightly higher rabies virus-specific antibody titers by ELISA and neutralization assay compared to those achieved with a second dose of the AdC68rab.gp vector. In either combination, priming with the AdHu5rab.gp vector elicited higher titers of antibodies than priming with the AdC68rab.gp vector. These results were compared with systemic prime boost regimens in which mice were immunized with a low dose (10⁵ pfu) of either AdHu5rab.gp or AdC68rab.gp vector. Mice were boosted two months later with the same dose of either homologous or heterologous vaccine carrier. Control groups did not receive the second dose of vaccine. Serum antibody titers to rabies virus analyzed two weeks after the booster immunization showed high VNA titers of 1:100 IU upon a single immunization with the AdHu5rab.gp vector. A second immunization with either the AdHu5rab.gp or the AdC68rab.gp vector failed to increase these titers. The AdC68rab.gp vector, on the other hand, induced at these low doses only modest VNA titers, which failed to increase upon booster immunization with the homologous construct, indicating that neutralizing antibodies to the vaccine carrier impaired uptake of the second vaccine dose. Booster immunization of AdC68rab.gp-immune mice with the AdHu5rab.gp vector, on the other hand, increased VNA titers to rabies virus dramatically. These data demonstrate the high susceptibility of systemic Ad vector immunization to interference by neutralizing antibodies to the vaccine carrier. The same groups of mice which were orally vaccinated in these studies were analyzed for isotypes of rabies virus specific antibodies in vaginal lavage fluids. An unexpected difference in the effect of homologous versus heterologous prime-boost vaccination became apparent. Upon priming, vaginal lavage fluids from mice fed the AdHu5rab.gp or the AdC68rab.gp vector contained antibodies to rabies virus that, by 2 weeks after vaccination, were mainly of the IgA isotype. Upon booster immunization of AdHu5rab.gp-primed mice with either the AdHu5rab.gp or the AdC68rab.gp vectors, mice developed within 2 weeks a pronounced IgG2a response that exceeded the IgA response. Two months after booster immunization, the rabies virus-specific antibodies in vaginal lavage fluids of mice vaccinated twice per os with AdHu5rab.gp virus reversed to a preponderance of the IgA isotype. In contrast, in AdHu5rab.gp-primed mice boosted with the AdC68rab.gp vector, vaginal antibodies to rabies virus remained dominated by antibodies of the IgG2a isotype although levels of IgA antibodies were also substantial. AdC68rab.gp-primed mice showed low levels of IgA in their vaginal lavage 2 weeks after booster immunization with either the homologous or the heterologous vaccine carrier and levels of IgG antibodies were marginal. After two months, mice developed a pronounced IgG2a response that exceeded the IgA response. This was particularly noticeable in mice vaccinated twice with the AdC68rab.gp vector. To further assess the apparent preference of the two viral vectors to differentially induce mucosal IgA versus IgG2a antibodies to the transgene product, mice were vaccinated in follow-up experiments with an increased dose of either vaccine given at 2×10⁷ pfu per os. One month later, mice were boosted with the same dose of the homologous vectors and vaginal titers and isotypes of antibodies to rabies were determined two months later. AdHuSrab.gp-vaccinated mice developed high levels of IgA to rabies virus fluids while this isotype was virtually absent in vaginal lavage from mice immunized twice with the AdC68rab.gp vector. Mice immunized twice with AdC68rab.gp contained predominantly rabies virus-specific antibodies of the IgG2a isotype. These results indicated oral homologous versus heterologous booster immunization with two Ad vaccine carriers had a distinct effect on the isotypes of vaginal antibodies as was observed upon pre-exposure to AdHuS virus. Heterologous primer-boost vaccination regardless of the sequence of the vaccine carriers resulted in a balanced ratio or IgA or IgG2a antibodies (IgA:IgG2a ˜0.8). Conversely, a double immunization with AdHu5rab.gp vector favored induction of vaginal IgA over IgG2a antibodies to rabies virus (IgA:IgG2a>2) while repeated oral application of the AdC68rab.gp vector strongly favored induction of vaginal IgG2a over IgA responses (IgA:IgG2a>0.3).

The unexpected efficacy of homologous oral prime boosting with the AdHuS vector was compatible with the observed lack of interference of pre-exposure to wild-type AdHu5 virus on the transgene product-specific antibody response to oral AdHu5rab.gp vaccination. These results indicate that, upon oral immunization, intestinal production of antibodies to the vaccine carrier is either low or short-lived and that pre-exposure or priming through intranasal or oral routes fails to affect uptake of the same vaccine carrier given several weeks later per os. It has been shown that intranasal immunization with the AdHu5 vector resulted in sustained antibody titers to the antigens of the vaccine carrier in fecal suspension (Xiang and Ertl (1999) supra). In similar studies conducted herein, mice vaccinated orally with 2×10⁷ or 2×10⁶ pfu of the AdHu5rab.gp vaccine had readily detectable antibody titers, of predominantly IgA, to the antigens of AdHu5 virus in fecal suspensions one month after the vaccination.

It has also been found that adenoviral vectors can be used in intranasal and oral immunization of newborn mice to induce antigen-specific antibody responses which are not impaired by maternally transferred antibodies to the vaccine carrier. The immune system of neonatal mammals is immature at birth and thus prone to the development of tolerance as is best exemplified by the unresponsiveness of newborn mice to allogenic lymphocytes. This can be circumvented by presenting the alloantigens by dendritic cells, indicating that the induction of neonatal tolerance in this system is a reflection of the relative lack of stimulator cells rather than an immaturity of the responding T cell populations (Ridge, et al. (1996) Science 271:1723). Other antigens if given at high doses have been shown to induce apparent tolerance of CD8+ T cells by favoring induction of Th2 responses (Sarzottii, et al. (1996) Science 271:1726; Forsthuber, et al. (1996) Science 271:1728). Immunization of neonates with a DNA vaccine expressing an antigen of malaria resulted in antigen-specific tolerance (Mor, et al. (1996) J. Clin. Invest. 98:2700), while the same vaccine induced a potent immune response in adult mice (Gardner, et al. (1996) J. Pharm. Sci. 85:1294). Applying the DNA vaccine with GM-CSF in the form of a genetic adjuvant evoked an immune response in neonates again suggestive of a primary defect of antigen presenting cells as the underlying mechanism for tolerance induction (Ishii, et al. (1999) Vaccine 18:703).

Neonates, despite the immaturity of their immune system, can respond to antigens. Neonatal lymphocytes secrete cytokines upon activation (Byun, et al. (1994) J. Immunol. 153:4862; Wu, et al. (1993) J. Immunol. 151:1938), although there is a relative deficiency in the production of IFN-gamma (Brysen, et al. (1980) Cell Immunol. 55:191), which favors induction of Th2-type immune responses. Ig⁻ pre-B cells that are more susceptible to tolerization are frequent in neonates (Nossal (1996) Annu. Rev. Immunol. 13:1); nevertheless, neonates also have mature B cells that secrete specific antibodies upon activation. Functional maturation of the mucosal immune system lags behind that of the systemic immune system (Spencer and MacDonald (1990) In: Ontogeny of the Immune System of the Gut. T. T. MacDonald, ed. CRC, Boca Raton, pp. 23-50; Nelson, et al. (1994) J. Exp. Med. 179:203; Griebel and Hein (1996) Immunol. Today 17:30), potentially to allow a window for induction of tolerance to harmless antigens such as normal gut flora, food, or air pollutants. As a consequence, gastrointestinal infections with pathogenic bacteria are the leading cause of infant death worldwide. Notwithstanding, 10-day-old mice can produce IgA antibodies to enteric infections (Cuff, et al. (1992) Vaccine Res. 1:175; Sheridan, et al. (1983) Infect. Immun. 39:917).

Using the AdHu5rab.gp vector, is has been shown that mice vaccinated systemically at birth developed protective antibody titers to rabies virus unless they had maternally transferred antibodies to the vaccine carrier (Wang, et al. (1996) Virology 228:278). Employing the AdHu5rab.gp vector as well as the E1-deleted AdC68 recombinant derived from a chimpanzee origin adenovirus for experiments disclosed herein, groups of 8-10 pups from naïve ICR dams were immunized within 24-48 hours after birth with 10⁷ pfu of AdHu5rab.gp or AdC68rab.gp virus given intranasally or orally. In some experiments, a control virus expressing GFP was included as a negative control. Pups were bled 3 weeks later and serum antibody titers and isotype profiles were determined by ELISA. Pups immunized with either of the recombinant Ad viruses expressing the rabies virus glycoprotein given through the mucosal routes developed serum antibodies to rabies virus. Such a response was not elicited by the control vector. Both recombinants gave comparable responses upon intranasal administration to neonates while upon oral immunization the AdHu5rab.gp vector elicited a higher antibody response compared with the AdC68rab.gp vector. The isotypes of antibodies to rabies virus were mixed in pups immunized orally or intranasally with the AdHu5rab.gp vector and composed of approximately equal levels of IgG1 and IgG2a. Upon oral or intranasal immunization, the AdC68rab.gp vector induced by 3 weeks of age a pronounced IgG1 response with no detectable IgG2a to the rabies virus antigen.

As mucosal delivery of vaccines to neonatal mice is technically challenging, a number of individual sera were tested from 3-week-old female pups immunized at birth with the AdC68rab.gp vaccine given either intranasally or orally. Although titers varied in individual pups, which may in part reflect inaccurate delivery of the vaccine dose to neonatal mice as well as genetic differences of the cohort of outbred pups, all of the pups had readily detectable titers to rabies virus. In orally immunized pups, these titers were remarkably stable and comparable when tested again from the same cohort of animals 6 months later. Titers had increased by then in intranasally vaccinated pups, indicating a delay in responsiveness or a longer persistence of the vaccine-encoded antigen upon airway delivery.

VNA titers tested 3 weeks after immunization of pups were low but detectable in all groups. VNA titers did not correlate with ELISA titers and were comparable in all of the groups except for the group immunized by the AdHu5rab.gp vector given intranasally. VNA titers to rabies virus correlate with protection and titers above 0.5 IU are known to prevent disease following a peripheral challenge with an otherwise lethal dose of rabies virus. VNA titers of pooled sera from all of the vaccinated groups exceeded the 0.5 IU benchmark.

Vaginal lavage fluid was harvested from 2-month-old female pups and tested for antibody isotypes to rabies virus to determine whether pups developed mucosally secreted antibodies upon neonatal immunization. Both AdHu5rab.gp and AdC68rab.gp vaccines given orally or intranasally resulted in vaginal antibodies to rabies virus. Isotypes were mixed with the AdHu5rab.gp vector, again showing a higher propensity to induce Th1-linked antibodies of the IgG2a isotype than the AdC68rab.gp vector. Intranasally AdC68rab.gp-vaccinated pups had substantial levels of rabies virus-specific IgA antibodies in their vaginal lavage fluids.

To analyze whether the induction of a rabies virus-specific antibody response upon oral delivery of the AdHu5rab.gp vector was affected by maternal transfer of antibodies to the vaccine carrier, female ICR dams were immunized intranasally with 10⁹ pfu of AdHu5 virus and bred to ICR males. Pups from naïve and AdHu5-immune dams were vaccinated within 24-48 hours after birth with 10⁷ pfu of AdHu5rab.gp virus given orally. Some pups from the immune dams were left unvaccinated and used to assess titers of maternally transferred antibodies to AdHu5 virus once pups were 3 weeks old. Unvaccinated pups had serum antibody titers to the AdHu5 virus that could be detected by ELISA, but were below those detectable by a neutralization assay (i.e., < 1/20) Titers were substantially lower than those found in their vaccinated dams that were bled and tested in parallel. The pooled sera from the dams had a neutralizing titer to AdHu5 virus of 1/160. Maternal antibodies to adenovirus were in part secreted at mucosal surfaces; as such, antibodies were detected in the vaginal lavage fluids of female pups born to AdHu5-immune dams.

AdHu5rab.gp-vaccinated pups were also bled at 3 weeks of age and vaginal lavage fluid was harvested at 6 weeks of age from female pups. Titers and isotypes of antibodies to rabies virus were tested by an ELISA and a neutralization assay from sera and by an ELISA from vaginal lavage fluids using pooled samples. Immunization of dams with AdHu5 virus did not impair the transgene product-specific antibody response detected in sera or vaginal lavage fluid of pups vaccinated orally with the AdHu5rab.gp vector but rather caused an increase in serum antibody titers to rabies virus as was shown by ELISA and by neutralization assay using pooled sera. This was assessed further by testing titers of individual pups by ELISA. The results confirmed that maternal antibodies had not reduced the antibody response of the pups to rabies virus. Preimmunization did not have a major effect on the isotype profile of the early B cell response to the rabies virus glycoprotein expressed by the AdHu5rab.gp vaccine although pups born to immune dams developed a slightly higher ratio of IgG2a:IgG1 ratio in sera and higher levels of IgA in vaginal lavage fluid compared with those born to naïve dams. Further, female pups born to naïve dams responded better to oral vaccination compared with male pups; this difference was statistically significant (p=0.01 for the 1/400 and p=0.006 for the 1/800 dilution of serum) and not observed in pups born to AdHu5 virus-immune dams (p=0.156 for the 1/400 dilution of serum and p=0.123 for the 1/800 dilution of serum). Male pups born to AdHu5-immune dams developed higher antibody titers than those born to naïve dams, although this difference did not reach statistical significance (p=0.057 at the 1/400 serum dilution and p=0.72 at the 1/800 serum dilution).

Pups born to naïve or AdHu5-immune dams that had been vaccinated orally at birth with the AdHu5rab.gp vector were fully protected against challenge with 10 mean lethal doses of rabies virus given intranasally 2 months later.

Therefore, these results indicate that oral immunization of neonatal subjects is remarkably efficient at inducing systemic and mucosal transgene product-specific antibodies and can circumvent interference by maternal Abs.

To further illustrate the present invention, it was demonstrated that oral immunization of adult mice with a simian E1-deleted Ad vector (termed AdC6) expressing a truncated form of gag of HIV-1 (Schneider, et al. (1997) J. Virol. 71:4892-4903) induces gag-specific CD8⁺ T cells.

BALB/c mice vaccinated orally with 10¹¹ virus particles of the AdC6gag37 vector were tested 2 and 4 weeks later for gag-specific CD8⁺ T cells derived from various tissues including spleens, Peyers' patches, the intraepithelial lymphocytes (IELs) and mesenteric lymph nodes by intracellular staining for IFN-gamma. CD8+ T cells that produced IFN-gamma in response to the gag peptide could readily be demonstrated in the lymphocyte populations of the spleen but not in any of the other tissues analyzed. Splenic frequencies of gag-specific CD8⁺ T cells were comparable at both time points.

To determine if the gag-specific CD8⁺ T cells provided protection in a surrogate challenge model, mice vaccinated orally with 10¹¹ virus particles of the AdC6gag37 vector as well as age-matched naïve control mice were injected 8 weeks later intraperitoneally with 10⁶ pfu of a vaccinia virus recombinant expressing gag. Vaccinia virus titers determined from paired ovaries of individual mice 5 days later showed a reduction in geometric mean titers in vaccinated as compared to unvaccinated mice. In the vaccinated group, two out of nine animals had undetectable titers of below 100 pfu per paired ovaries and the highest titer in this group was 1.2×10⁴ pfu. The geometric mean titer of the entire group was 432 pfu. All of the control animals had readily detectable titers ranging from 1.2×10⁴ to 5×10⁵ pfu, with a geometric mean titer of 1.1×10⁵ pfu. These results indicate that the oral immunization had induced partially protective immunity against this surrogate challenge.

Frequencies of gag-specific CD8⁺ T cells elicited by oral immunization with high doses (10¹¹ virus particles) of vector were well below those achieved by more modest doses of 10⁸-10¹⁰ virus particles given by intramuscular injection, which have been shown to be on average 10 times higher, i.e., in the range of 10-15% of all splenic CD8+ T cells (Fitzgerald, et al. (2003) supra). To increase frequencies achieved by oral immunization with adenoviral vectors, a series of prime-boost experiments were conducted. Groups of BALB/c mice were immunized orally with 10¹¹ virus particles of the AdC6gag37 virus. Ten weeks later mice were boosted again orally with the same dose of either the homologous simian adenoviral vector, or a heterologous Ad vector of either simian (AdC68) or human (AdHu5) origin. For comparison, additional groups of mice were immunized only once with the vector used for booster immunization. Splenocytes were tested 10 days later for frequencies of IFN-gamma-producing CD8⁺ T cells. Prime boosting with the homologous adenoviral vector (i.e., AdC6gag37 given twice) or with a heterologous simian adenoviral vector (AdC6gag37 followed by AdC68gag37) failed to increase frequencies of splenic gag-specific CD8⁺ T cells. In contrast, booster immunization with the AdHu5gag37 vector affected a more than five-fold increase in the CD8⁺ T cell response to the transgene product.

As an alternative, a heterologous prime-boost regimen based on oral priming with the AdC6gag37 vector followed 2 months later by systemic (i.e., intraperitoneal) booster immunization with a vaccinia virus recombinant expressing gag was analyzed. This regimen achieved high frequencies of splenic CD8⁺ T cells, which were ˜10-30-fold above those elicited by either vaccine given separately.

These results demonstrate that oral immunization with E1-deleted adenoviral vectors stimulates at albeit low frequencies transgene product-specific CD8⁺ T cells, which suffice to provide some protection against a surrogate challenge. Frequencies of gag-specific CD8⁺ T cells elicited by oral AdC6 vector vaccination can be dramatically increased by a subsequent systemic boost with a vaccinia virus recombinant expressing gag (VVgag). These results indicate that simian Ad vectors are suited for oral priming of transgene product-specific CD8⁺ T cells.

Various serotypes of adenovirus were also tested to demonstrate that an immune response could be generated with any adenovirus-based vaccine administered orally. Groups of 5 ICR mice were immunized either intranasally (i.n.) or by feeding (per os) with 10⁷ pfu of AdC7rab.gp or AdC68rab.gp. Animals were bled at 2 and 4 weeks and titers isotypes of antibodies were tested by ELISA. Vaginal lavage fluid was also analyzed at 2 and 4 weeks for antibody isotype. Serum antibody titers to the various adenovirus vectors were comparable for oral and i.n. vaccinated mice at both 2 and 4 weeks. Antibodies detected at 2 and 4 weeks in serum were predominantly of the IgG1, IgG2a and IgG2b isotypes for both mice vaccinated both orally or i.n. with the various adenoviral vectors. Similarly, antibodies detected in vaginal lavage fluids were of the IgA, IgG1, and IgG2b isotypes at both 2 and 4 weeks in mice vaccinated orally or i.n. with the various adenoviral vectors. These results indicate that adenoviruses and vectors derived from adenoviruses can in general be used to induce an immune response in a subject.

Accordingly, the present invention generally relates to methods of inducing an immune response to an antigen by orally administering nucleic acid sequences encoding said antigen in an adenoviral-based vector. Oral administration of an adenoviral vector containing a transgene product is useful when an individual has been pre-exposed to either a wild-type adenovirus (i.e., natural exposure), pre-exposed to a recombinant adenoviral vector (i.e., as part of a vaccine), or pre-exposed indirectly to an advenovirus (e.g., in utero with exposure to maternal antibodies). Further, oral administration of an adenoviral vector containing a transgene product is useful as the priming vaccine followed by a systemic boost vaccine as well as both the prime and boost vaccine.

In one method of the invention an immune response is induced to a transgene product encoded by an adenovirus vector in a subject pre-exposed to a first adenovirus or adenoviral vector via oral administration of a second adenovirus forming the basis of a subunit vaccine. By administering the second adenovirus vector orally, an immune response to the transgene product encoded by the said second adenovirus vector is induced. As used herein, an immune response is defined as a mucosal or systemic immune response characterized by induction of a measurable B cell response or elicitation of a T cell response (e.g., CD4+ or CD8+ T cells) which is brought about by exposure to an antigen (e.g., exogenous antigens expressed from an adenovirus). As used herein, the term adenovirus, when used alone is intended to mean a wild-type adenovirus. When the terms recombinant adenovirus or recombinant adenoviral vector or adenoviral vector are used, these terms generally refer to wild-type adenoviruses which have been modified using recombinant technology and can function as adenoviral vaccine carriers. Pre-exposure to a first adenovirus is intended to include natural exposure to a wild-type adenovirus as well as exposure resulting from vaccination using an adenoviral vector. As demonstrated herein, exposure to a first adenovirus or adenoviral vector by any route may induce an immune response to said first adenovirus or adenoviral vector. Interference by this first immune response to a second adenoviral vector used as a vaccine delivery vehicle can be overcome upon oral administration of the second adenoviral vector. The effectiveness of oral administration to mount a second immune response is such that the first adenovirus or adenoviral vector and the second adenoviral vector may or may not be of the same serotype or from the same animal origin.

Adenoviral vectors of the present invention are generally replication-defective, i.e., a vector that is unable to replicate autonomously in a host cell. Typically, the genome of a replication-defective adenoviral vector lacks at least the sequences which are necessary for replication of said adenoviral vector in a host cell. These regions can be eliminated in whole or in part, be rendered non-functional, or be substituted by other sequences, in particular by nucleic acid sequence encoding an antigen of interest. In general, a replication-defective adenoviral vector retains the sequences of its genome which are necessary for encapsidating the viral particles. However, such sequences may also be replaced or modified.

Adenoviruses and adenoviral vectors exist as various serotypes whose structure and properties differ. Of these serotypes, use of any adenovirus or adenoviral vector of human, chimpanzee, or other non-human animal origin is desirable. Adenoviruses or adenoviral vectors of animal origin which can be used are adenoviruses or adenoviral vectors of canine, bovine, murine, ovine, porcine, avian, caprine, guinea pig, fowl, fish, possum, deer or simian origin. In particular embodiments, the adenovirus or adenoviral vector of animal origin is a simian or canine adenovirus or adenoviral vector. In other embodiments, use is made of adenoviruses or adenoviral vectors of human, simian, canine or mixed origin. Particularly suitable adenoviruses or adenoviral vectors of human or animal origin are well-known to those of skill in the art.

In general, a replication-defective adenoviral vector of the invention contains inverted terminal repeats, an encapsidation sequence and a nucleic acid sequence of interest. Further, in the genome of an adenoviral vector of the invention, at least the E1 region is non-functional. A viral gene under consideration may be rendered non-functional by any technique known to the person skilled in the art, in particular by total removal, substitution, partial deletion or the addition of one or more bases to the genes under consideration. Such modifications can be achieved in vitro on isolated DNA or in situ, for example using techniques of genetic manipulation or by treatment with mutagenic agents. Other regions can also be modified, in particular the E3 region (WO95/02697), the E2 region (WO94/2938), the E4 region (WO94/28152, WO94/12649 and WO95/02697) and the L1-L5 regions (WO95/02697). An adenoviral vector, according to the present invention, can contain a deletion or multiple deletions, for example, a deletion in the E1 and E4 regions or a deletion in E1 and E3 regions. Further, an adenoviral vector of the invention can contain a deletion in the E1 region into which a nucleic acid of interest is inserted. The sequence of interest can alternatively be inserted into the E3 domain.

A nucleic acid sequence of particular interest for use as a transgene is one which encodes a product or an antigen to which an immune response is directed. Products or antigens which can be encoded by such sequences include, but are not limited to, antigenic epitopes or proteins from cancerous cells (e.g., tumor cell surface-specific proteins), viruses, fungi, bacteria, protozoa, mycoplasma or other proteins (e.g., aberrant proteins) that can be targeted by an immune response to benefit the afflicted individual. In one embodiment, the first adenoviral vector and second adenoviral vector encode the same transgene product. In an alternative embodiment, the first adenoviral vector and second adenoviral vector encode different transgene products. In particular embodiments, antigens are derived from enveloped or non-enveloped viruses. In accordance with this embodiment, antigens are derived from viruses including, but not limited to, those from the family Arenaviridae (e.g., Lymphocytic choriomeningitis virus), Arterivirus (e.g., Equine arteritis virus), Astroviridae (Human astrovirus 1), Birnaviridae (e.g., Infectious pancreatic necrosis virus, Infectious bursal disease virus), Bunyaviridae (e.g., California encephalitis virus Group), Caliciviridae (e.g., Caliciviruses), Coronaviridae (e.g., Human coronaviruses 299E and OC43), Deltavirus (e.g., Hepatitis delta virus), Filoviridae (e.g., Marburg virus, Ebola virus), Flaviviridae (e.g., Yellow fever virus group, Hepatitis C virus), Hepadnaviridae (e.g., Hepatitis B virus), Herpesviridae (e.g., Epstein-Bar virus, Simplexvirus, Varicellovirus, Cytomegalovirus, Roseolovirus, Lymphocryptovirus, Rhadinovirus), Orthomyxoviridae (e.g., Influenzavirus A, B, and C), Papovaviridae (e.g., Papillomavirus), Paramyxoviridae (e.g., Paramyxovirus such as human parainfluenza virus 1, Morbillivirus such as Measles virus, Rubulavirus such as Mumps virus, Pneumovirus such as Human respiratory syncytial virus), Picornaviridae (e.g., Rhinovirus such as Human rhinovirus 1A, Hepatovirus such Human hepatitis A virus, Human poliovirus, Cardiovirus such as Encephalomyocarditis virus, Aphthovirus such as Foot-and-mouth disease virus O, Coxsackie virus), Poxyiridae (e.g., Orthopoxvirus such as Variola virus or monkey poxvirus), Reoviridae (e.g., Rotavirus such as Groups A-F rotaviruses), Retroviridae (Primate lentivirus group such as human immunodeficiency virus 1 and 2), Rhabdoviridae (e.g., rabies virus), Togaviridae (e.g., Rubivirus such as Rubella virus), Human T-cell leukemia virus, Murine leukemia virus, Vesicular stomatitis virus, Wart virus, Blue tongue virus, Sendai virus, Feline leukemia virus, Simian virus 40, Mouse mammary tumor virus, or Dengue virus.

In a further embodiment, an antigen is derived from Streptococcus agalactiae, Legionella pneumophilia, Streptococcus pyogenes, Escherichia coli, Neisseria gonorrhosae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Candida albicans, Cryptococcus neoformans, Histoplasma capsulatum, Coccidioides immitis, Blastomyces dermatitidis, Aspergillus fumigatus, or Penicillium marneffei.

In a further embodiment, an antigen is an aberrant protein derived from a sequence which has been mutated. Such antigens include those expressed by tumor cells or aberrant proteins whose structure or solubility leads to the formation of an aggregation-prone product and cause disease. Exemplary aberrant proteins which can be encoded by the transgene of an orally-administered adenoviral vector of the present invention include, but are not limited to, Alzheimer's amyloid peptide (Aβ), SOD1, presenillin 1 and 2, α-synuclein, amyloid A, amyloid P, CFTR, transthyretin, amylin, lysozyme, gelsolin, p53, rhodopsin, insulin, insulin receptor, fibrillin, α-ketoacid dehydrogenase, collagen, keratin, PRNP, immunoglobulin light chain, atrial natriuretic peptide, seminal vesicle exocrine protein, β2-microglobulin, PrP, precalcitonin, ataxin 1, ataxin 2, ataxin 3, ataxin 6, ataxin 7, huntingtin, androgen receptor, CREB-binding protein, dentaorubral pallidoluysian atrophy-associated protein, maltose-binding protein, ABC transporter, glutathione-S-transferase, and thioredoxin.

Further, aberrant proteins encompass those which support the growth of an unwanted cell (e.g., tumor or fat cell). For example, such a protein can be produced by an endothelial cell that forms vessels (e.g., angiogenic factors) and/or provides nutrients to the unwanted cell.

Suitable nucleic acid sequences encoding antigens are well-known to those of skill in the art and can be identified from the GENBANK® or EMBL databases. The nucleic acid sequences can encode a protein, peptide, or epitope of an antigen and can have exogenous or endogenous expression control sequences, such as an origin of replication, a promoter, an enhancer, or necessary information processing sites, such as ribosome binding sites, RNA splice sites, polyadenylation sites, and transcriptional terminator sequences. Construction of such nucleic acid sequences is well-known in the art and is described further in Maniatis et al., Molecular Cloning: A Laboratory Manual, 2nd Ed., Cold Spring Harbor Laboratory, Cold Spring Harbor, N.Y. (1989). The sequence can be modified to optimize expression, change stability or retarget the antigen to alternative cellular compartments. Preferred expression control sequences may be promoters derived from metallothionine genes, actin genes, immunoglobulin genes, CMV, SV40, adenovirus, bovine papilloma virus, and the like. The adenoviral vectors can in addition to the antigen contain sequences that encode proteins with known immunomodulatory functions such as cytokine, pathogen-associated molecular patterns, chemokines, growth factors, and the like.

An adenoviral vector of the invention can be prepared as exemplified herein or by any technique known to one of skill in the art (see, for example, Levreto, et al. (1991) Gene 101:195; EP 185 573; Graham (1984) EMBO J. 3:2917). In particular, they can be prepared by homologous recombination between an adenovirus or adenoviral vector and a plasmid which carries, inter alia, the nucleic acid sequence of interest. The homologous recombination is effected following cotransfection of the said adenovirus or adenoviral vector and plasmid into an appropriate cell line. The cell line which is employed should be transformable by said elements, and contain the sequences which are able to complement the part of the genome of the replication-defective adenoviral vector, preferably in integrated form in order to avoid the risks of recombination. Examples of cell lines which can be used include the human embryonic kidney cell line 293 (Graham, et al. (1977) J. Gen. Virol. 36:59) which contains, in particular, integrated into its genome, the left-hand part of the genome of an Ad5 adenovirus (12%), or cell lines which are able to complement the E1 and E4 functions (see, e.g., WO94/26914 and WO95/02697). Subsequently, the or adenoviral vectors which have multiplied are recovered and purified using standard molecular biological techniques, as illustrated in the examples.

Alternatively, the vector can be derived from a so-called molecular clone wherein the adenoviral genome including the foreign sequences is first generated in bacterial plasmids. The virus is then rescued in tissue culture. Methods for generating and using molecular clones are well-described and routine to one of skill in the art. A molecular clone is generally desirable in generating an adenoviral vector originating from a species other than humans if the purpose of said vector is for use as a vaccine carrier in humans.

The present invention also includes pharmaceutical compositions containing one or more adenoviral vectors dispersed in a physiologically acceptable medium, which is, in general, buffered to physiologically normal pH. Such pharmaceutical compositions, in accordance with the present invention are formulated for administration by oral routes. The pharmaceutical composition or pharmaceutical preparation contains an efficacious dose of an adenoviral vector and a pharmaceutically acceptable carrier. Oral administration of the pharmaceutical composition can be in the form of pills, tablets, lacquered tablets, coated tablets, granules, hard and soft gelatin capsules, solutions, paste, gel, solid or semi-solid form, syrups, emulsions, suspensions or aerosol mixtures.

The selected pharmaceutically acceptable carrier can be an inert inorganic and/or organic carrier substance and/or additive. For the production of pills, tablets, coated tablets and hard gelatin capsules, the pharmaceutically acceptable carrier can include lactose, cornstarch or derivatives thereof, talc, stearic acid or its salts, and the like. Pharmaceutically acceptable carriers for soft gelatin capsules include, for example, fats, waxes, semisolid and liquid polyols, natural or hardened oils, and the like. Suitable carriers for the production of solutions, emulsions, or syrups include, but are not limited to, water, alcohols, glycerol, polyols, sucrose, glucose, and vegetable oils. Suitable carriers for microcapsules include copolymers of glycolic acid and lactic acid.

In addition to an adenoviral vector and a pharmaceutically acceptable carrier, the pharmaceutical composition can contain an additive or auxiliary substance. Exemplary additives include, for example, fillers, disintegrants, binders, lubricants, wetting agents, stabilizers, emulsifiers, preservatives, sweeteners, colorants, flavorings, aromatizers, thickeners, diluents, buffer substances, solvents, solubilizers, salts for altering the osmotic pressure, coating agents or antioxidants. A generally recognized compendium of methods and ingredients of pharmaceutical compositions is Remington: The Science and Practice of Pharmacy, Alfonso R. Gennaro, editor, 20th ed. Lippingcott Williams & Wilkins: Philadelphia, Pa., 2000.

Further, the adenoviral vector can be administered with an adjuvant to enhance a subject's T cell response to the antigen. Examples of such adjuvants include, but are not limited to, aluminum salts; Incomplete Freund's adjuvant; threonyl and n-butyl derivatives of muramyl dipeptide; lipophilic derivatives of muramyl tripeptide; monophosphoryl lipid A; 3′-de-o-acetylated monophosphoryl lipid A; cholera toxin; phosphorothionated oligodeoxynucleotides with CpG motifs and adjuvants disclosed in U.S. Pat. No. 6,558,670. Alternatively, the adjuvant can be encoded by sequences inserted into the adenoviral genome and can be present in either the first and/or second adenoviral vector in combination with the transgene product.

Dosage and administration are adjusted to provide sufficient levels of the adenoviral vector or to maintain the desired effect of providing protecting immunity, preventing or reducing signs or symptoms of a disease or infection, or reducing severity of a disease or infection. Factors which can be taken into account include the severity of the disease state, general health of the subject, age, weight, and gender of the subject, diet, time and frequency of administration, drug combination(s), reaction sensitivities, and tolerance/response to therapy. Such factors can be assessed by a physician or qualified medical professional and the amount adjusted accordingly. In one embodiment, an effective amount of an adenoviral vector is administered such that a measurable immune response is induced to the transgene product upon exposure to said adenoviral vector containing nucleic acid sequences encoding the transgene product. A measurable B cell response can be determined by, for example, production of antibodies to the antigen, and elicitation of a T cell response can be determined, for example, by measuring the production of cytokines, e.g., IFN-gamma, IL-2, IL-4, IL-5, or IL-10.

Generally, the recombinant adenoviruses according to the invention are formulated and administered in the form of doses of between about 10⁴ and about 10¹¹ pfu or between about 10⁵ to 10¹² viral particles. Alternatively, the doses of adenoviral vector are from about 10⁵ to about 10¹¹ pfu or about 10⁶ to about 10¹² viral particles. The term pfu (plaque-forming unit) corresponds to the infective power of a suspension of virions and is determined by infecting an appropriate cell culture and measuring, generally after 7-14 days, the number of plaques of infected cells. Alternatively, such assays can be based on limiting dilution and on the use of genetic methods such as PCR reactions to detect the viral genome or transcripts thereof. The techniques for determining the pfu titer of a viral solution are well documented in the literature.

In cases where administration of the first and second adenoviral vectors are part of a vaccination protocol, the first adenoviral vector or priming adenoviral vector can be administered orally, parenterally injected (such as by intraperitoneal, subcutaneous, or intramuscular injection), or topically using well-known formulations and amounts to induce an immune response to said first adenoviral vector and antigen(s) encoded thereby. Topical application can be carried out by intranasal administration (e.g., by use of dropper, swab, or inhaler which deposits a pharmaceutical formulation intranasally) or direct contact with the skin such as in a cream, ointment, or gel.

Alternatively, in accordance with another method of the invention, an immune response is induced to a transgene product encoded by an adenovirus vector by oral priming with an effective amount of the adenoviral vector and subsequently systemically boosting with an effective amount of an adenoviral vector encoding for the same transgene product. Protocols for oral priming are disclosed herein and protocols for systemic boosting are well-known in to the general practitioner.

The first adenoviral vector can be given as a single dose schedule, or preferably in a multiple dose schedule. A multiple dose schedule is one in which a primary course of administration may be with 1-10 separate doses, followed by other doses (i.e., second adenoviral or boost adenoviral vector) given at subsequent time intervals required to maintain and or reinforce the immune response, for example, at 1 to 4 months for a second dose, and if needed, a subsequent dose(s) after several months. The dosage regimen will also, at least in part, be determined by the need of the individual and be dependent upon the judgement of the practitioner.

The present invention offers a novel and very efficient means for inducing an immune response to an adenoviral vector in a subject and can be used as part of a vaccine or therapy for adult or infant humans or other animals such as sheep, cattle, domestic animals (e.g., dogs and cats), and fish.

Thus, another method of the invention relates to inducing an immune response in an infant by orally administering to the infant an effective amount of an adenoviral vector encoding a transgene product so that an immune response to the transgene product is induced. As will be appreciated the one of skill in the art, the first exposure of an infant to immunity to an adenovirus is indirect via the mother. Adenovirus-specific immunity provided to the infant via the mother or the mother's milk can be circumvented by oral administration of an effective amount of an adenoviral vector encoding a transgene product. As used herein, an infant is intended to include newborn mammals having circulating maternal antibodies against an adenovirus. For example, where reference is made to humans, a neonate or infant is generally less than 12 months old; for canines, the neonate is generally less than 16 weeks old; for felines, the neonate is generally less than 16 weeks old. However, in general, this method may be employed on all mammalian infants under 1 year of age. Based on this information, the skilled artisan can readily determine the appropriate age range for the selected mammalian neonate vaccinee.

The present invention also relates to a method for inducing a mucosal immune response to an antigen. The method involves orally administering an effective amount of a first adenoviral vector containing nucleic acid sequences encoding an antigen, and subsequently orally administering an effective amount of a second adenoviral vector containing said nucleic acid sequences encoding said antigen. In this method of the invention, an effective amount of adenoviral vector containing an antigen is administered in an amount which results in a measurable mucosal immune response. A mucosal immune response involves the production of mucosa-related IgA and IgG and a complement of T cells with mucosa-specific regulatory or effector properties and provides for host defense at the mucosal surfaces. For a more complete review of the mucosal immune system see Strober and James, “The Mucosal Immune System” In Basic & Clinical Immunology 8th Edition eds Stites, Terr, Parslow, (Appleton & Lange, 1994), pgs 541-551.

The invention is described in greater detail by the following non-limiting examples.

### EXAMPLE 1

### Mice, Cell Lines and Viruses

Mice. Female inbred mice and outbred ICR mice were used at 6-12 weeks of age. Female 6-8-week-old inbred mice were purchased from Jackson Laboratory (Bar Harbor, Me.). Adult female and male ICR mice, as well as time-pregnant ICR mice, were purchased from Charles River Breeding Laboratories (Boston, Mass.).Cell Lines. BHK-21 and 293 cells were maintained in Dulbeccos' modified Eagles medium (DMEM) supplemented with 10% fetal bovine serum (FBS) and antibiotics.

E1-transfected 293 cells, TK⁻ 143B (TK⁻) cells and HeLa cells were propagated in DMEM supplemented with glutamine, sodium pyruvate, non-essential amino acids, HEPES buffer, antibiotic and 10% FBS.

Viral Recombinants. AdHu5rab.gp recombinant, E-1 deleted Ad recombinant of the human serotype 5 expressing the glycoprotein of the ERA strain of rabies virus is well-known in the art (Belyakov, et al. (1999) Proc. Natl. Acad. Sci. USA 96:4512-4517). E1-deleted AdC68rab.gp vaccine expressing the same transgene product in a simian Ad virus vector is also well-known in the art (Farina, et al. (2001) supra; Xiang, et al. (2002) supra). Viral recombinants, including AdHu5 and AdC68 vectors expressing green fluorescent protein (GFP) as well as wild-type AdHu5 virus, were propagated and titrated on 293 cells. Viral recombinants were harvested by freeze thawing of infected 293 cells followed by pelleting of the cellular debris. In addition, the AdHu5 virus was purified by CsCl centrifugation and virus particles per milliliter were determined by spectrophotometry at 260 nm according to standard methods (Farina, et al. (2001) supra).

Dosing of the recombinant Ad vectors was in terms of numbers of plaque forming units (pfu) or virus particles (vps). Dosing of wild-type Ad was in terms of numbers of pfu or vps; the latter so that induction of antibodies against the Ad antigens by defective virus particles was taken into account.

The ERA strain of rabies virus was grown on BHK-21 cells, purified by gradient centrifugation and inactivated by treatment with beta-propiolactone (BPL) (Wiktor, et al. (1973) WHO Monogr. Ser. 23:101-123). The protein content of the inactivated virus (ERA-BPL) was determined and adjusted to 1 mg/ml. The rabies stain CVS-11 used for challenge was propagated and titrated on BHK-21 cells (Wiktor, et al. (1978) Dev. Biol. Stand. 40:171-178). For neonatal immunization studies, mice were challenged with the challenge virus strain CVS-N2C of rabies (Morimoto, et al. (1999) J. Virol. 73:510), a variant of CVS-24 strain which is closely related to the ERA strain but is highly virulent in mice. The virus was derived from brains of neonatally infected ICR mice and titrated by intranasal challenge of young adult ICR mice.

A vaccinia virus recombinant expressing gag of HIV-1 clade B was obtained from the NIH AIDS Research and Reference Reagent Program (Germantown, Md.). The virus was propagated on HeLa cells. Cleared cell-free lysate of infected cells was titrated on TK⁻ cells to determine pfu. E1-deleted adenoviral recombinants of the human serotype 5, or simian serotypes (AdC6 and AdC68) expressing a codon-optimized truncated form of gag of HIV-1, gag37 (Fitzgerald, et al. (2003) J. Immunol. 170:1416-1422), were propagated on E1-transfected 293 cells. Recombinant virus was purified by CsCl gradient centrifugation, virus particles and pfu/ml were determined in accordance with standard methods (Farina, et al. (2001) supra).

### EXAMPLE 2

### Immunization and Challenge of Mice

Mice were immunized once or twice with various doses, indicated in pfu, of the AdHu5 or AdC68 constructs given per os or intramuscularly (i.m.). Mice immunized with an Ad viral recombinant expressing a viral antigen not derived from rabies virus are unable to induce rabies virus-specific antibodies (Xiang, et al. (2002) supra). Thus, this control was not included in the experiments conducted herein. Oral immunization with 10⁶ pfu of the AdHu5rab.gp vaccine fails to induce serum antibody titers to rabies virus. Thus, the vaccination procedure was modified by applying the vaccines with a feeding tube to ensure swallowing rather than inhalation or spillage of the vaccine. Furthermore, the vaccine was diluted in a buffered salt solution rather than in saline. Mice were immunized with wild-type AdHu5 virus given intranasally or i.m. Mice were challenged with 10 mean lethal doses (LD₅₀) of the CVS-11 strain of rabies virus injected directly into the brain. Experiments were conducted 2-5 times in groups of 5 to 8 mice to ensure reproducibility.

When using inbred mice, groups of five mice were immunized at 6-8 weeks of age with recombinant viruses diluted in 100 μL of phosphate buffered saline (PBS). The vaccines were applied orally with the help of a small feeding tube. For intramuscular immunizations the vaccines were diluted in 50 μL of saline, vaccinia virus was given intraperitoneally (i.p.). For some experiments, mice were boosted 2-3 months after the first immunization.

For neonatal immunization studies, adult female ICR mice were immunized once with 10⁹ pfu of AdHu5 virus given intranasally and 2 days later cohoused with males. Males were separated from pregnant females before birth of the pups. Pups were immunized within 24-48 hours after birth intranasally or orally with 10⁷ pfu of AdHu5rab.gp, AdC68rab.gp, or an AdC68 control vector expressing GFP (AdC68GFP). Alternatively, pups were left unvaccinated to establish titers of maternally transferred antibodies.

### EXAMPLE 3

### Preparation of Samples

Blood was harvested by retro-orbital puncture. Sera were prepared and heat-inactivated at 56° C. for 30 minutes. Sera were tested for rabies virus-neutralization starting at a 1:5 dilution and for neutralization of AdHu5 virus starting at a 1:20 dilution. Analysis was conducted by ELISA starting with a 1:200 dilution. Antibody isotypes were tested with a 1:800 dilution of sera. Vaginal lavage fluid was harvested by rinsing the vaginal cavity three times with 50 μl of saline for a final volume of 150 μl. The sample was centrifuged at 5000 or 10,000 rpm for 5 or 10 minutes to remove debris. Vaginal lavage fluid was titrated starting at a dilution of 1:2; antibody isotypes were determined with a 1:8 dilution (Xiang, et al. (1999) J. Immunol. 162:6716-6723). Feces was collected and suspended at 50 mg/mL in PBS containing 1% NaN₃. After a one hour incubation at room temperature, samples were vortexed and debris was removed by centrifugation at 14,000 rpm in an EPPENDORF® centrifuge (Xiang and Ertl (1999) supra). Samples were tested for antibody titers starting at a 1:2 dilution and for isotypes at a 1:5 dilution. Neonatal samples were analyzed by ELISA starting with a 1:200 dilution and antibody isotypes were tested with a 1:400 dilution of sera or a 1:2 dilution of vaginal lavage fluid (Xiang, et al. (1994) supra).

Spleens, cervical and mesenteric lymph nodes and Peyers' patches were harvested 18 to 72 hours after oral immunization.

Single cells were prepared from spleens and mesenteric lymph nodes by gently rubbing the organs against a stainless steel mesh. Clumps were removed by filtration through a nylon filter. For preparation of IELs, the intestines were collected and washed. Peyer's patches were removed. The intestines were cut longitudinally, and upon removal of fecal content cut into small pieces. IELs were isolated by gentle stirring of the intestines at 37° C. for 30 minutes in medium containing antibiotics. IELs were then purified first through a tea strainer, then through a loosely packed glass wool column and then by centrifugation through a PERCOLL™ step gradient.

### EXAMPLE 4

### Enzyme-Linked Immunoadsorbent Assay

Sera, vaginal lavage and fecal suspensions were tested on rabies virus-coated plates using well-established methods (Xiang, et al. (1999) supra). Briefly, round-bottom microtiter plate wells were coated overnight with 0.2 μg of ERA-BPL virus or purified AdHu5 virus diluted in 100 μl of coating buffer (15 mM Na₂CO3, 35 mM NaHCO₃, 3 mM Na₂N, pH 9.6). Plates were subsequently treated for 24 hours with PBS containing 3% bovine serum albumin (BSA). The following day plates were washed two times with 150 μl of PBS for 24 hours, dried and stored at −20° C. Sera were serially diluted in PBS containing 3% BSA. The different dilutions of sera were incubated in duplicate at 100 μl per well on the ERA-BPL coated plates for 1 hour at 4° C. Fecal suspensions and vaginal lavage fluids were incubated overnight. Plates were washed five times with PBS and treated with an alkaline phosphatase-conjugated, goat anti-mouse antibody for 1 hour at 4° C. Plates were washed and incubated for 20 minutes with the substrate (10 mg d-nitrophenyl phosphate disodium dissolved in 10 ml of 1 mM MgCl₂, 3 mM NaN₃, 0.9 M diethanolamine, pH 9.8). Plates were then measured in an automated ELISA reader at 405 nm. Isotypes of antibodies to rabies virus were tested on ERA-BPL-coated plates with the CALBIOCHEM® isotyping kit, which has comparable sensitivity for different antibody isotypes (Vos, et al. (2001) supra). Isotype ELISAs were read at 450 nm.

### EXAMPLE 5

### Virus Neutralization Assay

Sera were tested on BHK-21 cells for neutralization of CVS-11 virus, which is closely related, antigenically, to the ERA virus (Wiktor (1973) supra). Sera were tested on 293 cells for neutralization of AdHu5 virus or Ad recombinants expressing GFP by a plaque reduction assay (Farina, et al. (2001) supra).

### EXAMPLE 6

### Reverse Transcription Polymerase Chain Reaction

Mice were sacrificed and lymphoid tissues were harvested and disrupted by a polytron probe in a solution of TRI-REAGENT® (MRC, Cincinnati, Ohio). RNA was isolated from individual samples as recommended by the manufacturer. Briefly, 100 μl of BCP solution (MRC, Cincinnati, Ohio) was added to each sample. The aqueous phase was transferred to fresh tubes, and RNA was precipitated by isopropanol, washed with 70% ethanol and resuspended in DEPC-treated water (AMBION®, Inc., Houston, Tex.). DNA was removed by treatment with DNAse (AMBION®, Inc. Houston, Tex.) for 30 minutes at 37° C. DNAse was removed with the DNAse removal kit (AMBION®, Inc., Houston, Tex.). Complementary DNA (cDNA) was synthesized from RNA samples with M-MLV Reverse Transcriptase (Life Technologies, Inc., Rockville, Mass.). Samples were amplified for rabies virus glycoprotein and glyceraldehyde 3-phosphate dehydrogenase (GAPDH) cDNAs using the following primers: rab.gp-forward, 5′-AAA GCA TTT CCG CCC AAC AC-3′ (SEQ ID NO:1); rab.gp-reverse, 5′-GGT TAC TGG AGC AGT AGG TAG A-3′ (SEQ ID NO:2); GAPD-forward, 5′-GGT GAA GGT CGG TGT GAA CGG ATT T-3′ (SEQ ID NO:3); and GAPDH-reverse 5′-AAT GCC AAA GTT GTC ATG GAT GAC C-3′ (SEQ ID NO:4). PCR conditions for all genes included an initial denaturation at 94° C. for 5 minutes and 40 cycles of: denaturation at 94° C. for 1 minutes, annealing at 55° C. for 2 minutes and extension at 72° C. for 3 minutes. The amplicons were separated by electrophoresis on a 1% agarose gel, visualized by ethidium bromide, and analyzed on a FLUORIMAGER™ SI (Vistra Fluorescence, Sunnyvale, Calif.).

### EXAMPLE 7

### Peptides

The Ala-Met-Gln-Met-Leu-Lys-Glu-Thr-Ile peptide (SEQ ID NO:5) (Doe and Walker (1996) AIDS 10:793-794) that carries the immunodominant MHC class I epitope of gag for mice of the H-2 d haplotype, and the control peptide 31D delineated from the nucleoprotein of rabies virus (Ertl, et al. (1989) J. Virol. 63:2885-2892), were synthesized using standard methods. The peptides were purified by high-pressure liquid chromatography and sequence-verified by mass spectrometry. Peptides were diluted in DMSO to a concentration of 1 mg/mL and stored at −20° C.

### EXAMPLE 8

### Intracellular Cytokine Staining

Splenocytes (1×10⁶ per sample) were cultured for 5 hours at 37° C. in 96-well round-bottom microtiter plate wells in DMEM supplemented with 2% FBS and 10-6 M 2-mercaptoethanol. Brefeldin A (GOLGIPLUG™, PHARMINGEN™, San Diego, Calif.) was added at 1 μL/mL. The Ala-Met-Gln-Met-Leu-Lys-Glu-Thr-Ile peptide (SEQ ID NO:5) peptide was used at a concentration of 1.5 μg/mL. Control cells were incubated with an unrelated peptide or without peptide. After washing, cells were incubated for 30 minutes at 4° C. with 25 μL of a 1:100 dilution of a FITC-labeled antibody to mouse CD8 (PHARMINGEN™). They were washed again and permeabilized in 1×CYTOFIX/CYTOPERM™ (PHARMINGEN™) for 20 minutes at 4° C., washed three times with PERM/WASHT™ (PHARMINGEN™), and incubated in the same buffer for 30 minutes at 4° C. with 25 μL of a 1:100 dilution of a PE-labeled antibody to mouse IFN-gamma (PHARMINGEN™). After washing, cells were examined by two-color flow cytometry using an EPICS® Elite XL (Beckman-Coulter Inc., Miami, Fla.) and data were analyzed by WinMDi software. Cells incubated with a peptide derived from the nucleoprotein of rabies virus served as controls. Additional controls included lymphocytes from naïve or sham-vaccinated mice.

### EXAMPLE 9

### Vaccinia Virus Challenge

Mice (9-10 per group) immunized with the AdC6gag37 vector given orally and age-matched naïve control mice were injected intraperitoneally with 10⁶ pfu of the vaccinia gag recombinant virus. Paired ovaries from individual mice harvested 5 days later were homogenized in 1 mL of medium, freeze-thawed three times and cell-free supernatant was titrated on confluent monolayers of TK⁻ cells. Cells were stained 36-48 hours later with crystal violet and plaques were counted under low magnification.

### EXAMPLE 10

### Statistical Analysis

Experiments were conducted at least twice using at least five mice per group. Sera were tested by ELISA or neutralization assay in duplicates or triplicates. Results show the means±SDs. Significance was calculated using Student's t test for two to three dilutions. Data with p values below 0.05 were considered to reflect a statistical significance.

What is claimed is:

1. A method for inducing an immune response to a transgene product in a subject pre-exposed to an adenovirus or adenoviral vector comprising orally administering to a subject, that has been exposed to a first adenovirus or adenoviral vector, an effective amount of a second adenoviral vector encoding a transgene product so that an immune response to the transgene product is induced.

2. The method of claim 1, wherein the first adenoviral vector and the second adenoviral vector encode the same transgene product.

3. The method of claim 1, wherein the first adenoviral vector and the second adenoviral vector encode different transgene products.

4. The method of claim 1, wherein the transgene product is an antigenic epitope or protein from a cancer cell, virus, fungus, bacterium, protozoa, mycoplasma or aberrant protein.

5. The method of claim 1, wherein the first adenovirus is a wild-type virus and the second adenovirus comprises a vaccine.

6. The method of claim 1, wherein the first adenoviral vector and the second adenoviral vector comprise a vaccine.

7. The method of claim 5, wherein the second adenoviral vector further encodes an adjuvant.

8. The method of claim 6, wherein the first adenoviral vector or second adenoviral vector further encode an adjuvant.

9. A method for inducing an immune response to a transgene product comprising orally administering to a subject an effective amount of a first adenoviral vector encoding a transgene product and subsequently systemically administering to the subject an effective amount of a second adenoviral vector encoding said transgene product.

10. The method of claim 9, wherein the transgene product is an antigenic epitope or protein from a cancer cell, virus, fungus, bacterium, protozoa, mycoplasma or aberrant protein.

11. The method of claim 9, wherein the first and second adenoviral vector comprise a vaccine.

12. The method of claim 11, wherein the first or second adenoviral vector further encode an adjuvant.

13. A method for inducing an immune response in an infant comprising orally administering to an infant an effective amount of an adenoviral vector encoding a transgene product so that an immune response to the transgene product is induced.

14. The method of claim 13, wherein the transgene product is an antigenic epitope or protein from a virus, fungus, bacterium, protozoa, mycoplasma or aberrant protein.

15. The method of claim 13, wherein the adenoviral vector comprises a vaccine.

16. The method of claim 15, wherein the adenoviral vector further encodes an adjuvant.

17. A method for inducing a mucosal immune response to an antigen comprising:
orally administering an effective amount of a first adenoviral vector containing nucleic acid sequences encoding an antigen, and
orally administering an effective amount of a second adenoviral vector containing said nucleic acid sequences encoding said antigen,
so that a mucosal immune response is induced.

18. The method of claim 17, wherein the first adenoviral vector and the second adenoviral vector encode the same transgene product.

19. The method of claim 17, wherein the first adenoviral vector and the second adenoviral vector encode different transgene products.

20. The method of claim 17, wherein the antigen is from a cancer cell, virus, fungus, bacterium, protozoa, mycoplasma or aberrant protein.

21. The method of claim 17, wherein the first adenoviral vector and second adenoviral vector comprise a vaccine.

22. The method of claim 19, wherein the first or second adenoviral vector further encode an adjuvant.


SEQUENCE LISTING (5 sequences)

SEQ ID NO: 1 - DNA, 20 bp, Artificial Sequence
  Synthetic oligonucleotide primer
aaagcatttccgcccaacac

SEQ ID NO: 2 - DNA, 22 bp, Artificial Sequence
  Synthetic oligonucleotide primer
ggttactggagcagtaggtaga

SEQ ID NO: 3 - DNA, 25 bp, Artificial Sequence
  Synthetic oligonucleotide primer
ggtgaaggtcggtgtgaacggattt

SEQ ID NO: 4 - DNA, 25 bp, Artificial Sequence
  Synthetic oligonucleotide primer
aatgccaaagttgtcatggatgacc

SEQ ID NO: 5 - protein, 9 aa, Artificial Sequence
  Synthetic peptide
AMQMLKETI